(19) United States
(12) Patent Application Publication
(10) Pub. No.: US 2023/0330039 A1
(43) Pub. Date: Oct. 19, 2023

(54) FOOD SUPPLEMENT AND FOOD

(71) Applicant: PM-International AG, Schengen (LU)

(72) Inventors: Rolf Sorg, Schengen (LU); Wilhelm Messer, Bad Dürkheim (DE); Christian Lauinger, Ettlingen (DE)

(21) Appl. No.: 18/008,012

(22) PCT Filed: Jul. 10, 2020
(86) PCT No.: PCT/EP2020/069513

§ 371 Date: Dec. 2, 2022

(30) Foreign Application Priority Data
Jun. 9, 2020 (DE) 10 2020 115 238.8

(51) Int. Cl. A61K 31/07; A23L 33/12; A23L 33/105; A23L 33/125; A23L 2/39; A23L 2/60; A23L 2/68; A61K 31/202; A61K 31/122; A61K 9/00; A61K 36/258

(52) U.S. Cl. CPC A61K 31/07; A23L 33/12; A23L 33/105; A23L 33/125; A23L 2/39; A23L 2/60; A23L 2/68; A61K 31/202; A61K 31/122; A61K 9/0095; A61K 36/258

(57) ABSTRACT

The invention relates to a powdered composition containing per 100 g of the composition
25 mg to 250 mg of lutein,
2 mg to 12 mg of zeaxanthin,
1000 mg to 5000 mg of docosahexaenoic acid,
100 mg to 1200 mg of coenzyme Q10 and
30 g to 90 g of one or more excipients selected from thickeners, emulsifiers and stabilizers.

The invention relates to dietary supplements and food products. Like food products, dietary supplements are commonly administered orally. To improve absorption of the ingredients in the gastrointestinal tract, to simplify ingestion of the dietary supplement, and to avoid complications during ingestion, dietary supplements are readily taken as an aqueous solution. Even with food products, taking them in liquid form is often preferred. To reduce microbial infestation and to increase chemical stability, dietary supplements are often offered as effervescent tablets or powders, which are then dispersed in water by the user to produce a drinkable solution.

Inadequate in many cases is the dissolution behavior of the ingredients of the effervescent tablets or powder and the visual appearance and taste of the solution produced. Also unsatisfactory in some dietary supplements is insufficient bioavailability of valuable ingredients. It is also disadvantageous that many dietary supplements are not at all designed to meet the specific needs of certain consumers. Some dietary supplements contain a wide variety of ingredients. Thus, some consumers consume ingredients that they do not need at all. Other ingredients are only important for special target groups and are not added to most dietary supplements to avoid overdosing consumers who do not need these ingredients or need them in smaller quantities, or to be able to offer a product that is affordable for as many people as possible.

It is also desirable to improve the chemical and microbial stability of conventional dietary supplements and food products.

There is therefore a need for improved dietary supplements and food products that are versatile and can be used according to individual needs. In particular, there is a need for dietary supplements that are specifically geared to the needs of people aged 50 and over and that can be taken as a solution together with another dietary supplement or food product according to individual need and in an easily dosed manner.

As a dietary supplement or food product, it would therefore be desirable to have a composition that dissolves as quickly and completely as possible in water to form a healthy, drinkable solution with an appealing appearance and good taste, that allows a high bioavailability of the ingredients, that supplies people from about the age of 50 in particular with the ingredients that are especially important for them, and that can be taken as a solution together with another dietary supplement or food product according to individual needs and in an easily dosed manner.

The present invention addresses this need and is intended to overcome the above disadvantages of the prior art and solves at least part of the problem by providing a powdered composition containing, per 100 g of the composition, from 25 mg to 250 mg of lutein, from 2 mg to 12 mg of zeaxanthin, from 1000 mg to 5000 mg of docosahexaenoic acid, from 100 mg to 1200 mg of coenzyme Q10, and from 30 g to 90 g of one or more excipients selected from thickeners, emulsifiers and stabilizers.

Lutein is a lipophilic xanthophyll for which administration has been shown to have beneficial effects in age-related macular degeneration, cataract, and dry eyes. Lutein also shows a positive effect on cognitive functions and brain function. Preferably, the lutein is added to the composition in the form of a powder further containing zeaxanthin, starch, and dried glucose syrup, wherein said amounts in mg refer to lutein in pure form. The lutein may also be added to the composition in pure form.

Zeaxanthin is also a lipophilic xanthophyll useful for the prevention of age-related macular degeneration. Preferably, the zeaxanthin is added to the composition in the form of a powder further containing lutein, starch, and dried glucose syrup, wherein said amounts in mg refer to zeaxanthin in pure form. The zeaxanthin may also be added to the composition in pure form.

Lutein and zeaxanthin appear to play a particularly important role in protecting the retina and vision. In particular, they appear to counteract damage caused by blue light (i.e., light of a wavelength between 400 and 500 nm). Such blue light is emitted by the sun, of course, but also by LEDs and/or the screens of televisions, computers and mobile phones.

The unsaturated omega-3 fatty acid docosahexaenoic acid has numerous beneficial effects, especially on the cardiovascular system, the immune system and the central nervous system. The composition contains one omega-3 unsaturated fatty acid (docosahexaenoic acid) or a multiple thereof (e.g. docosahexaenoic acid and eicosapentaenoic acid) added to the composition, preferably in the form of a powder, wherein the powder is a spray-dried powder containing refined algal oil (e.g. from Schizochytrium sp.), corn syrup solids and starch, wherein said amounts in mg refer to docosahexaenoic acid in pure form. Due to the special formulation of the algae oil or unsaturated fatty acid(s), a powdered, free-flowing and thus easily dosed composition can be obtained.

Coenzyme Q10 is a fat-soluble compound with generally poor bioavailability. The compound is important for energy balance and as a free radical scavenger. An adequate supply of coenzyme Q10 has positive effects on the heart, the natural aging process and physical performance. Preferably, coenzyme Q10 is added to the composition according to the invention in the form of a complex of γ-cyclodextrin and coenzyme Q10 (weight ratio in the complex circa 3:1), with the amounts given in mg referring to the coenzyme Q10 in pure form. Complexation can increase the water solubility and bioavailability of coenzyme Q10.

The one or more excipients promote the composition to dissolve quickly and completely in water to provide a drinkable solution with an appealing appearance and good taste. Further, the excipient or excipients allow the composition to be in powder form so that the powdered composition can be easily dosed according to individual needs and can be taken together with another dietary supplement as a solution.

By merely adding the powdered composition to water or an aqueous solution in a drinking vessel and stirring briefly, a solution or at least a drinkable dispersion can be prepared at room temperature (about 20° C.). A suspension is considered a solution or drinkable dispersion in the sense of the invention as long as the suspended parts are dispersed in a drinkable manner, i.e., are substantially drunk along with it and do not remain at the bottom of the drinking vessel. Compositions in which the ingredients substantially sediment to the bottom of the drinking vessel and are not simply drunk along are not considered to be a solution or drinkable dispersion in the sense of the invention. An advantage of the formulation as a water-soluble powder is that the ingredients have a high stability in dry form and can enter the bloodstream particularly quickly when used from solution.

It is also advantageous that established dietary supplements and/or food products can be individualized by the powdered composition according to the invention and thus, for example, persons from about the age of 50 can be supplied particularly well and specifically.

The composition according to the invention is a dietary supplement and/or a food product.

The verbs “contain” and “comprise” and their conjugations also include the verb “consist of” and its conjugations.

Preferred embodiments of the invention may be combined with each other, as long as nothing else arises from the context. Preferred embodiments are also indicated in the claims.

Preferably, the one or more excipients are selected from gum arabic, alginic acid, alginates, agar-agar, carrageenan, locust bean gum, guar gum, tragacanth, xanthan gum, karaya gum, tara gum, gellan gum, starches, starch phosphates and gelatin. Gum arabic is particularly preferred.

Preferably, the composition contains as the one or more excipients selected from thickeners, emulsifiers and stabilizers, gum arabic. In other words, particularly preferably, the powdered composition contains, per 100 g of the composition, 25 mg to 250 mg of lutein, 2 mg to 12 mg of zeaxanthin, 1000 mg to 5000 mg of docosahexaenoic acid, 100 mg to 1200 mg of coenzyme Q10, and 30 g to 90 g, more preferably 40 g to 80 g, particularly preferably 50 g to 70 g, of gum arabic.

Gum arabic is particularly suitable for imparting to the powdered composition the dissolving behavior and drinkability already described. In particular, gum arabic acts simultaneously as a thickener, emulsifier and stabilizer. As a result, lipophilic substances can also be dissolved or dispersed in a drinkable form and ingested.

Furthermore, gum arabic in particular can be used to provide a powder with good flow properties and thus good dosability.

Preferably, then, the composition is water-soluble, or at least all of the ingredients can be dispersed (potably) in water (without significant sedimentation).

Preferably, the composition contains 1 g to 10 g, more preferably 1 g to 6 g, particularly preferably 2 g to 4 g, of sugar per 100 g of the composition. In other words, the sugar content of the composition is 1 g to 10 g, more preferably 1 g to 6 g, particularly preferably 2 g to 4 g, per 100 g of the composition. The sugar content results from sugar added in pure form and/or sugar included in extracts (e.g., ginseng extract) and/or as a carrier (e.g., for lutein, omega-3 fatty acids), and added to the composition with the extract (e.g., ginseng extract) or as a carrier (e.g., for lutein, omega-3 fatty acids). The sugar content leads to a good taste and the sugar itself is water soluble.

Preferably, the composition contains at least one steviol glycoside, whereby the taste can be improved. In other words, preferably the composition contains 0.01 g to 1 g, more preferably 0.1 g to 0.6 g, particularly preferably 0.2 to 0.4 g, of one or more steviol glycosides per 100 g of the composition. By combining sugar and the at least one steviol glycoside, a particularly healthy product can be obtained, since the sugar content of the composition can be reduced, but the solution still contains sugar as an energy carrier or cosubstrate for transporters in the gastrointestinal system.

Preferably, the composition contains 0.1 g to 10 g or 0.3 g to 7 g or 0.4 g to 6 g of red ginseng (Panax ginseng) extract per 100 g of the composition, wherein the extract contains at least 5% by weight of ginsenosides. Preferably, the extract is an extract from the root. Preferably, the extractant is water. Preferably, the extract is added to the composition in the form of a spray-dried powder with a maltodextrin base.

Preferably, the composition contains 0.5 g to 5 g, more preferably 1 g to 3 g, of acidifying agent, preferably citric acid, per 100 g of the composition. The acidifying agent, in particular citric acid, can also contribute to the special taste experience, counteract the oxidation of certain ingredients in aqueous solution and improve the solubility of certain ingredients in aqueous solution.

Artificial sweeteners are not required and in a preferred embodiment the composition does not contain artificial sweeteners. Artificial sweeteners within the meaning of the invention include in particular acesulfame (E 950), advantame (E 969), aspartame (E 951), aspartame-acesulfame salt (E 962), cyclamate (E 952), neohesperidin (E 959), neotame (E 961), saccharin (E 954) and sucralose (E 955). Steviol glycosides, e.g. stevioside (E 960), are not considered artificial sweeteners in the sense of the invention.

The composition is microbially stable. Artificial preservatives are not required and preferably the composition does not contain artificial preservatives.

Preferably, the composition contains 25 mg to 200 mg, more preferably 40 mg to 130 mg, particularly preferably 60 mg to 90 mg, of eicosapentaenoic acid per 100 g of the composition. Like docosahexaenoic acid, the unsaturated omega-3 fatty acid eicosapentaenoic acid exhibits numerous beneficial effects, particularly on the cardiovascular system, the immune system and the central nervous system.

Preferably, the composition contains 150 mg to 3000 mg, more preferably 500 mg to 1500 mg, particularly preferably 750 mg to 1250 mg, of isoflavones, preferably soyaisoflavones, per 100 g of the composition. Isoflavones and especially isoflavones from soy (soyaisoflavones) are used for menopausal symptoms, osteoporosis, hypertension, high cholesterol, memory problems, incontinence and overactive bladder. Soyaisoflavones preferentially bind to and transactivate estrogen receptor 13 rather than estrogen receptor a, mimicking the effects of estrogen in some tissues and antagonizing the effects of estrogen in other tissues. Estrogen receptor a is the predominant receptor in the breast and uterus, and estrogen receptor 13 is the predominant receptor in the cardiovascular system, genitourinary tract, and bone. This could enable anti-estrogenic effects in reproductive tissues and reduce the risk of developing hormone-associated cancers in the breast and prostate. In contrast, estrogenic effects in other tissues may help maintain bone density and improve blood lipid profiles. Preferably, isoflavones are extracted from soybean germs, preferably using ethanol. In particular, soy isoflavones comprise the glycosides genistin, daidzin and glycitein, and their aglycones genistein, daidzein and glycitein. In other words, the composition preferably contains 150 mg to 3000 mg, more preferably 500 mg to 1500 mg, particularly preferably 750 mg to 1250 mg, of one or more isoflavones selected from genistin, daidzin and glycitein and genistein, daidzein and glycitein. In one embodiment, the aglycones genistein, daidzein and glycitein are preferred because absorption is faster and bioavailability is higher.

Intestinal bacteria can convert glycosides into aglycones, improving absorption in the gastrointestinal tract. Intestinal bacteria can also convert daidzein to equol. Equol exhibits greater estrogenic activity at the estrogen receptor 13 than daidzein, so equol is thought to mediate some, if not most, of the health benefits of isoflavones. Equol [7-hydroxy-3-(4′-hydroxyphenyl)-chroman] is a nonsteroidal estrogen. It has the molecular formula C151-11403 and a molecular weight of 242.27 daltons. Studies measuring urinary excretion of equol after soy consumption show that equol is produced by about 25% to 30% of the adult population in Western countries. In contrast, 50% to 60% of adults living in Asian countries and/or living in Western countries and following a vegetarian diet produce equol. Various intestinal bacteria cultured in vitro can biotransform daidzein to S-(-)equol or related intermediates. This is consistent with the observation that equol producers have a higher complexity of the gut microbiome. In contrast, antibiotic use appears to decrease aglycone production. Soyaisoflavone ingestion for a period longer than 16 weeks may positively promote the growth of gut bacteria that support equol production.

Preferably, the composition contains 1 g to 10 g, more preferably 1 g to 6 g, particularly preferably 2 g to 4 g, of saturated fatty acids per 100 g of the composition. Thus, the product is low in saturated fatty acids and particularly healthy.

Preferably, the composition contains 50 mg to 175 mg, more preferably 75 mg to 125 mg, of lutein per 100 g of the composition.

Preferably, the composition contains 3 mg to 10 mg, more preferably 5 mg to 7 mg, of zeaxanthin per 100 g of the composition.

Preferably, the composition contains 1500 mg to 3500 mg, more preferably 2000 mg to 3000 mg, of docosahexaenoic acid per 100 g of the composition.

Preferably, the composition contains 300 mg to 1000 mg, more preferably 500 mg to 700 mg, of coenzyme Q10 per 100 g of the composition.

It is also an object of the invention to provide a kit containing a powdered composition according to the invention (“first composition”) and a second powdered composition, wherein the second powdered composition is a dietary supplement and/or food product. In the kit, the first and second compositions are present spatially separated from each other, e.g., the first and second compositions are each packaged in a container within the kit. Advantageously, established dietary supplements and/or food products can be individualized by the powdered composition according to the invention and thus, for example, persons from about the age of 50 can be supplied particularly well and in a targeted manner.

It is also an object of the invention to use the composition according to the invention or the kit according to the invention as a dietary supplement and/or food product, wherein from 1 g to 10 g, more preferably from 2 g to 8 g, particularly preferably from 4 g to 6 g, of the (first) composition is taken in one day. Preferably, the composition is taken by a person who is at least 50 years old. Preferably, the composition is taken orally as a solution or drinkable dispersion. In a preferred embodiment, the use is not a medical use.

It is also an object of the invention to provide a method of preparing a solution for oral administration. In this process, the powdered composition according to the invention and optionally a second powdered composition which is a dietary supplement and/or food product is dissolved or dispersed in water to produce a drinkable solution. In this process, 3 g to 7 g, preferably 4 g to 6 g, of the (first) composition according to the invention are added to about 100 mL to 300 mL, preferably 150 mL to 250 mL, of water and mixed to obtain a solution or at least a drinkable dispersion. Preferably, the dissolution is carried out at 10° C. to 30° C., more preferably at 15° C. to 25° C. or approximately at room temperature.

It is also an object of the invention to provide a method for preparing a composition according to the invention, comprising the mixing of 25 mg to 250 mg of lutein, 2 mg to 12 mg of zeaxanthin, 1000 mg to 5000 mg of docosahexaenoic acid, 100 mg to 1200 mg of coenzyme Q10 and 30 g to 90 g of one or more excipients selected from thickeners, emulsifiers and stabilizers, and optionally one or more ingredients selected from 0.1 g to 10 g of red ginseng (Panax ginseng) extract, 0.5 g to 5 g of acidifying agent, preferably citric acid, 25 mg to 200 mg of eicosapentaenoic acid, 150 mg to 3000 mg, of isoflavones, preferably soyaisoflavones, and 0.01 g to 1 g of one or more steviol glycosides.

In a preferred embodiment of the kit, the kit contains as a second powdered composition the dietary supplement or food product described below. The following explanations up to the example part apply exclusively to the second composition.

The second powdered composition is a powdered composition for preparing a solution for oral administration, wherein the composition contains, per 100 g, 1 g to 85 g of at least one monosaccharide, 0.18 g to 0.70 g of at least one steviol glycoside, 1.5 g to 10 g of citric acid, 0.3 g to 3 g of vitamin C, at least two other vitamins, and an extract of green tea (Camellia sinensis). Preferably, the second powdered composition dissolves as quickly and completely as possible in water to provide a drinkable solution, with the solution having an appealing appearance and taste. Preferably, the second powdered composition provides high bioavailability of the ingredients and/or improves oxygenation in the human body and/or reduces oxidative stress and/or increases the concentration of endogenous nitric oxide.

The at least one monosaccharide leads to the rapid dissolution of the powder in water and good taste. The at least one steviol glycoside also contributes to the good taste. By combining at least one monosaccharide and at least one steviol glycoside, a particularly healthy product is obtained, since the amount of monosaccharides can be reduced and the solution still contains at least one monosaccharide as an energy carrier. The citric acid also contributes to the special taste experience. Artificial sweeteners are not required and preferably the second composition does not contain artificial sweeteners.

The second composition is microbially stable. Artificial preservatives are not required and preferably the second composition does not contain artificial preservatives.

The vitamin C and the at least two other vitamins are vitamins that are not part of the green tea extract. Preferably, the vitamin C and the at least two other vitamins are added as pure substances.

In preferred embodiments, the second composition contains, per 100 g, 5 g to 80 g of the at least one monosaccharide, 0.19 g to 0.60 g of the at least one steviol glycoside, 2.5 g to 7.0 g of citric acid, and 0.4 g to 2.5 g of vitamin C.

Preferably, the second composition contains, per 100 g, from 4.14 mg to 3000 mg of the extract of green tea (Camellia sinensis), preferably from leaves of green tea. The extract contains valuable polyphenols, catechins and contributes to the special taste of the solution.

Preferably, the second composition contains
a concentrate of broccoli (Brassica oleracea italica), in particular of seeds, sprouts and/or shoots of broccoli, and/or
a concentrate of broccoli (Brassica oleracea italica), in particular from florets and/or stems of broccoli, and/or
an extract of onion (Allium cepa, in particular Allium cepa alliaceae), in particular from the bulb of the onion, and/or
an extract of apple (Malus domestica), in particular of apple fruit, in particular of peels of the apple fruit, and/or
an extract of acerola (Malpighia glabra, in particular Malpighia glabra linne), in particular the fruit of acerola, and/or
a concentrate of tomato (Lycopersicon esculentum), in particular from the fruit of the tomato, and/or
a concentrate of turmeric (Curcuma longa), in particular from the root of turmeric, and/or
a concentrate of garlic (Allium sativum), in particular from the garlic clove, and/or
a concentrate of basil (Ocimum basilicum), in particular of basil leaves, and/or
a concentrate of oregano (Origanum vulgare), in particular of leaves of oregano, and/or
a concentrate of the cinnamon tree (Cinnamonum cassia), in particular from the bark (without cork) of the cinnamon tree, and/or
a concentrate of carrot (Dacus carota, in particular Dacus carota sativa), in particular from the root of the carrot, and/or
a concentrate of elderberry (Sambucus nigra), in particular from the fruit of the elderberry, and/or
an extract of currant (Ribes), in particular of the blackcurrant (Ribes nigrum), in particular of the fruit of the blackcurrant, and/or
a concentrate of blueberry (Vaccinium), in particular from the fruit of the blueberry, and/or
a concentrate of raspberry (Rubus idaeus), in particular from the fruit of the raspberry, and/or
a concentrate of blackberry (Rubus spp.), in particular from the fruit of the blackberry, and/or
a concentrate of chokeberry (Aronia, in particular Aronia melanocarpa), in particular from the fruit of the chokeberry, and/or
a concentrate of spinach (Spinacia oleracea), in particular from the leaves of the spinach, and/or
a concentrate of cherry (Prunus avium), in particular from the cherry fruit, and/or
a concentrate of Brussels sprouts (Brassica oleracea gemmifera), in particular florets of Brussels sprouts.

Preferably, the second composition contains a total of 0.10 g to 0.75 g of these extracts and/or concentrates per 100 g of the composition.

Preferably, the second composition contains
a concentrate of broccoli (Brassica oleracea italica), in particular of seeds, sprouts and/or shoots of broccoli, and
a concentrate of broccoli (Brassica oleracea italica), in particular florets and/or stems of broccoli, and
an extract of onion (Allium cepa, in particular Allium cepa alliaceae), in particular the bulb of the onion, and
an extract of apple (Malus domestica), in particular of apple fruit, in particular of peels of the apple fruit, and
an extract of acerola (Malpighia glabra, in particular Malpighia glabra linne), in particular the fruit of acerola, and
a concentrate of tomato (Lycopersicon esculentum), in particular from the fruit of the tomato, and
a concentrate of turmeric (Curcuma longa), in particular from the root of turmeric, and
a concentrate of garlic (Allium sativum), especially from the garlic clove, and
a concentrate of basil (Ocimum basilicum), in particular of basil leaves, and
a concentrate of oregano (Origanum vulgare), especially leaves of oregano, and
a concentrate of the cinnamon tree (Cinnamonum cassia), in particular from the bark (without cork) of the cinnamon tree, and
a concentrate of carrot (Dacus carota, in particular Dacus carota sativa), in particular from the root of the carrot, and
a concentrate of elderberry (Sambucus nigra), in particular from the fruit of the elderberry, and
an extract of currant (Ribes), in particular of blackcurrant (Ribes nigrum), in particular of the fruit of the blackcurrant, and
a concentrate of blueberry (Vaccinium), in particular from the fruit of the blueberry, and
a concentrate of raspberry (Rubus idaeus), in particular from the fruit of the raspberry, and
a concentrate of blackberry (Rubus spp.), in particular from the fruit of the blackberry, and
a concentrate of chokeberry (Aronia, in particular Aronia melanocarpa), in particular from the fruit of the chokeberry, and
a concentrate of spinach (Spinacia oleracea), in particular from the leaves of the spinach, and
a concentrate of cherry (Prunus avium), in particular from the cherry fruit, and
a concentrate of Brussels sprouts (Brassica oleracea gemmifera), in particular florets of Brussels sprouts.

Preferably, the second composition contains a total of 0.10 g to 0.75 g of these extracts and concentrates per 100 g of the composition.

The term “extract” in reference to the second embodiment means a dehydrated extract from corresponding plant parts, wherein the extract is preferably obtained by means of an organic solvent and/or water and with a drug-extract ratio between 120:1 and 10:1. A “concentrate” means a dehydrated product from corresponding plant parts. Extracts and concentrates from fruits, leaves, bulbs, bark (excluding cork), roots, rhizomes, tubers, seeds, sprouts, shoots, florets, and stems provide high levels of valuable plant secondary metabolites such as polyphenols, depending on the plant species. Polyphenols are aromatic compounds that contain two or more hydroxyl groups directly attached to an aromatic ring and are classified as plant secondary metabolites. Polyphenols exhibit various health-promoting effects; in particular, some polyphenols have antioxidant effects. The concentrates and extracts of various plants provide a variety of health-promoting plant secondary metabolites, vitamins and polyphenols. Such a second composition may increase the bioavailability of the compounds contained in the second composition or a solution containing the first and second compositions. Therefore, the second composition exhibits a particularly health-promoting effect and provides particularly good support for a balanced diet. Furthermore, it was found that the addition of the extracts and/or concentrates has a positive effect on the dissolving behavior of the powder in water and the taste of the solution.

Preferably, the second composition contains, per 100 g, in each case 4 mg to 200 mg, preferably 5 mg to 50 mg, of one or more ingredients selected from an extract of the pepper (Piper nigrum) and an extract of the ginger (Zingiber officinale). Preferably, the second composition contains, per 100 g, 4 mg to 200 mg, preferably 5 mg to 50 mg, of an extract from the pepper (Piper nigrum), 0.25 mg to 2 g, preferably 0.45 mg to 1.5 g, of a curcumin compound, and 4 mg to 200 mg, preferably 5 mg to 50 mg, of an extract from the ginger (Zingiber officinale). The curcumin compound may be a curcumin-cyclodextrin complex, particularly a curcumin-gamma-cyclodextrin complex. Preferably, the extract from ginger is an extract from the rhizome of ginger (Zingiber officinale). These substances, even in such small doses, act as bioenhancers and increase the bioavailability of the ingredients of the composition. The improvement in bioavailability can be exploited by dissolving the first and second compositions in a glass of water even for ingredients that are present only in the first composition. Thus, the second composition can enhance the bioavailability of the first composition.

Preferably, the second composition contains, per 100 g, 2.3 mg to 5 g, more preferably 4.14 mg to 3 g, of extract from coffee (Coffea arabica), preferably from the bean of the coffee.

Preferably, the second composition contains, per 100 g, from 0.025 mg to 2 g, more preferably from 0.045 mg to 1.5 g, of extract from sour cherry (Prunus cerasus), preferably from the cherry of the sour cherry.

Preferably, the second composition contains, per 100 g, from 0.025 mg to 2 g, more preferably from 0.045 mg to 1.5 g, of extract from cabbage (Brassica oleracea acephala), preferably from leaves of cabbage.

Preferably, the second composition contains, per 100 g, from 0.25 mg to 2 g, more preferably from 0.45 mg to 1.5 g, of extract from turmeric (Curcuma longa), preferably from the root of turmeric.

Preferably, the second composition contains, per 100 g, from 0.025 mg to 2 g, more preferably from 0.045 mg to 1.5 g, of extract from broccoli (Brassica oleracea italica), preferably from the floret of broccoli.

Preferably, the second composition contains, per 100 g, from 0.025 mg to 2 g, more preferably from 0.045 mg to 1.5 g, of extract from blueberry (Vaccinium corymbosum), preferably from the fruit of the blueberry.

Preferably, the second composition contains an extract of coffee (Coffea arabica), preferably from the bean of the coffee, an extract of sour cherry (Prunus cerasus), preferably from the cherry of the sour cherry, an extract of cabbage (Brassica oleracea acephala), preferably from leaves of the cabbage, an extract of turmeric (Curcuma longa), preferably from the root of the turmeric, an extract of broccoli (Brassica oleracea italica), preferably from the floret of broccoli, an extract of blueberry (Vaccinium corymbosum), preferably from the fruit of blueberry, and the extract of green tea (Camellia sinensis), preferably from leaves of green tea, wherein the second composition contains, per 100 g, from 0.01 g to 5 g in total of these extracts. These extracts, especially in combination, can increase the concentration of endogenous nitric oxide.

Preferably, the second composition contains 100 mg to 1000 mg of caffeine per 100 g of the composition. The caffeine may be derived from a guarana extract.

In one embodiment of the second composition, the at least two further vitamins are selected from B vitamins (B vitamin embodiment). Preferably, the second composition in this embodiment contains 1 g to 85 g, more preferably 5 to 80 g, of the at least one monosaccharide per 100 g of the composition. Preferably, the second composition in this embodiment contains 0.19 g to 0.60 g of the at least one steviol glycoside per 100 g of the composition. Preferably, the second composition in this embodiment contains 2.5 g to 7.0 g of citric acid per 100 g of the composition. Preferably, per 100 g of the composition in this embodiment, the second composition further contains 50 mg to 200 mg of algae powder and optionally 3 g to 12 g, more preferably 4 g to 10 g, of guarana extract. The guarana extract may contribute, among other things, caffeine. Preferably, the algae powder is a powder of brown algae. Preferably, in this embodiment, the second composition contains dextrose as the at least one monosaccharide. In this embodiment, per 100 g of the composition, the second composition preferably contains as B vitamins 100 mg to 1500 mg of niacin, 20 mg to 270 mg of pantothenic acid, 5 mg to 100 mg of pyridoxine, 4 mg to 72 mg of riboflavin, 5 mg to 70 mg of thiamine, 10 μg to 10 mg of cyanocobolamine, 0.5 mg to 7 mg of biotin, and 0.5 mg to 12 mg of folic acid. Dextrose dissolves very quickly in water and, in combination with the other ingredients, gives the solution its special taste. Furthermore, dextrose improves the absorption of certain ingredients. In particular, this embodiment helps to improve the oxygen supply to the tissues.

In an alternative embodiment of the second composition, the at least two further vitamins are selected from vitamins A and E (vitamins A and E embodiment). Preferably, the second composition in this embodiment contains 1500 μg to 4000 μg of retinol equivalents and 60 mg to 120 mg of α-tocopherol equivalents per 100 g of the composition. Preferably, the second composition in this embodiment contains 5 g to 80 g, more preferably 5 g to 70 g, particularly preferably 5 g to 42 g, of the at least one monosaccharide per 100 g of the composition. Preferably, the second composition in this embodiment contains from 0.19 g to 0.60 g of the at least one steviol glycoside per 100 g of the composition. Preferably, the second composition in this embodiment contains from 2.0 g to 7.0 g of citric acid per 100 g of the composition. Preferably, the second composition in this embodiment contains fructose as the at least one monosaccharide. Preferably, the second composition in this embodiment contains 200 μg to 350 μg of selenium per 100 g of the composition. Preferably, the second composition in this embodiment contains 35 g to 85 g of dietary fiber per 100 g of the composition. The dietary fibers are preferably vegetable fibers from oats, pea, rice and/or apple. Fructose dissolves very quickly in water and, in combination with the other ingredients, gives the solution its beneficial taste. It is amazing that this particular second composition can be used to produce a solution despite the high fiber content. This embodiment is particularly beneficial for intestinal health and stimulates the immune system. The ingredients of the second (and first) composition can be absorbed even better in this variant. In this alternative embodiment of the second composition, it is also preferred that the composition contains digestive enzymes (amylase, protease, lactase, cellulase, and lipase) and a curcumin-gamma-cyclodextrin complex. In this alternative embodiment of the second composition, it is also preferred that the composition contains gram positive non-spore forming bacteria, such as (Lactobacillus acidophilus, Lactobacillus reuteri) and/or gram positive spore forming bacteria, such as Bacillus species.

The second composition may further contain excipients such as beet powder (concentrate of beet juice, maltodextrin), gum arabic, pectin and/or guar gum.

With respect to the use of the second composition in a kit as a dietary supplement and/or food product, it is preferred that from 1.25 g to 2.25 g of the B vitamin embodiment of the second composition is taken three times daily, or alternatively from 8 g to 16 g of the vitamins A and E embodiment of the second composition is taken once daily. The ingestion is oral, preferably as a solution or drinkable dispersion.

With respect to the method for preparing a solution for oral administration, it is preferred that, in the B vitamin embodiment of the second composition, 1.25 g to 2.25 g of the second composition is dissolved (or potably dispersed) in 20 mL to 100 mL, preferably circa 40 mL, of water and, in the vitamins A and E embodiment of the second composition, 8 g to 16 g of the second composition is dissolved (or potably dispersed) in 100 mL to 300 mL, preferably circa 180 mL, of water. Preferably, the dissolving is carried out at 10° C. to 30° C., more preferably at 15° C. to 25° C. or circa room temperature. Preferably, the second composition is first dissolved in water and then the first composition is added and dissolved.

### EXAMPLES

The invention is explained below by means of an example.

A (first) powdered composition according to the invention is obtained by mixing 25 g of a spray-dried powder containing refined algal oil of Schizochytrium sp., corn syrup solids and starch, 3 g of a complex of γ-cyclodextrin and coenzyme Q10, 0.9 g of a red ginseng extract containing at least 5% ginsenosides, 0.3 g of steviol glycosides, 1.5 g of citric acid, 2 g of a powder containing lutein, zeaxanthin, starch and dried glucose syrup, 2.5 g of a soy extract containing isoflavones and powdered gum arabic per 100 g obtained. 100 g of the composition contains the following ingredients, among others:

| | | |
|---|---|---|
| Coenzyme Q10 | 660 | mg |
| Lutein | 110 | mg |
| Zeaxanthin | 6.6 | mg |
| Isoflavones | 1050 | mg |
| Docosahexaenoic acid | 2625 | mg |
| Eicosapentaenoic acid | 78 | mg |

The nutritional table contains the following values for 100 g of the composition:

| | | |
|---|---|---|
| Calorific value | 287 | kcal |
| Fat content | 8 | g |
| thereof saturated | 2.7 | g |
| Carbohydrate content | 26 | g |
| thereof sugar | 3 | g |

By merely adding the powdered composition to water or an aqueous solution in a drinking vessel and stirring briefly, a solution or at least a drinkable dispersion can be prepared at room temperature (about 20° C.).

The solution has an attractive appearance and good taste, provides especially people from about 50 years of age with ingredients that are particularly important for them. The powdered composition is free-flowing, according to individual needs and easy to dose and can be taken together with a dietary supplement or food as a solution.

This (“first”) composition according to the invention can be provided in a kit together with a second composition. Two example formulations for such second compositions are given below.

Example formulation for the second composition according to B vitamin embodiment of the second composition. 100 g of the powdered composition contain:
Vitamin C, 2.5 g;
Niacin, 0.34 g;
Caffeine, 0.8 g;
Pantothenic acid, 0.05 g;
Pyridoxine, 0.02 g;
Thiamine, 0.02 g;
Riboflavin, 0.02 mg;
Cyanocobolamine, 10 μg;
Folic acid, 2.0 mg;
Biotin, 1.5 mg;
Citric acid, 7.5 g;
Guarana extract, 9.5 g;
Steviol glycosides, 0.4 g;
Algae powder, 0.1 g;
Green tea extract (Camellia sinensis), 5 mg;
Beet powder (concentrate of beet juice, maltodextrin), 1 g;
Dextrose ad 100 g.

Example formulation for the second composition according to vitamins A and E embodiment of the second composition. 100 g of the powdered composition contain:
Steviol glycosides, 0.3 g;
Citric acid, 3.5 g;
Vitamin C, 1.5 g;
Green tea extract (Camellia sinensis), 0.08 g;
2000 μg retinol equivalents;
70 mg α-tocopherol equivalents;
250 μg selenium;
46 g dietary fiber;
Lactobacillus acidophilus, Lactobacillus reuteri, 0.3 g;
Amylase, protease, lactase, cellulase and lipase, 0.3 g;
Fructose ad 100 g.

The powdered second compositions dissolve quickly in water and form drinkable solutions without artificial preservatives or sweeteners. The solutions have an appealing appearance and taste, provide high bioavailability of the ingredients, and improve the supply of oxygen, essential vitamins, polyphenols, and other substances to the human body. In these solutions of the second compositions, the first powdered compositions according to the invention dissolve quickly.

1. A powdered composition, the powdered composition comprising per 100 g of the powdered composition
25 mg to 250 mg of lutein,
2 mg to 12 mg of zeaxanthin,
1000 mg to 5000 mg of docosahexaenoic acid,
100 mg to 1200 mg of coenzyme Q10 and
30 g to 90 g of one or more excipients selected from thickeners, emulsifiers and stabilizers.

2. The powdered composition according to claim 1, wherein the one or more excipients are selected from gum arabic, alginic acid, alginates, agar-agar, carrageenan, locust bean gum, guar gum, tragacanth, xanthan gum, karaya gum, tara gum, gellan, starches, starch phosphates and gelatin.

3. The powdered composition according to claim 1, wherein the powdered composition contains as excipient 30 g to 90 g of gum Arabic per 100 g of the powdered composition.

4. The powdered composition according to claim 1, wherein the powdered composition is water-soluble.

5. The powdered composition according to claim 1, wherein the powdered composition contains 1 g to 10 g of sugar per 100 g of the powdered composition.

6. The powdered composition according to claim 1, wherein the powdered composition contains 0.1 g to 10 g of red ginseng (Panax ginseng) extract per 100 g of the powdered composition.

7. The powdered composition according to claim 1, wherein the powdered composition contains 0.5 g to 5 g of acidifying agent per 100 g of the powdered composition.

8. The powdered composition according to claim 1, wherein the powdered composition contains 25 mg to 200 mg of eicosapentaenoic acid per 100 g of the powdered composition.

9. The powdered composition according to claim 1, wherein the powdered composition contains 150 mg to 3000 mg of isoflavones per 100 g of the powdered composition.

10. The powdered composition according to claim 1, wherein the powdered composition contains 0.01 g to 1 g of one or more steviol glycosides per 100 g of the powdered composition.

11. The powdered composition according to claim 1, wherein the powdered composition contains 1 g to 10 g of saturated fatty acids per 100 g of the powdered composition.

12. The powdered composition according to claim 1, wherein the powdered composition contains
50 mg to 175 mg of lutein and/or
3 mg to 10 mg of zeaxanthin and/or
1500 mg to 3500 mg of docosahexaenoic acid and/or
300 mg to 1000 mg of coenzyme Q10 per 100 g of the powdered composition.

13. A kit, the kit comprising a first powdered composition which is the powder composition according to claim 1 and a second powdered composition which is a supplement and/or food.

14. A method of use of the powdered composition according to claim 1, the method comprising taking from 1 g to 10 g of the powdered composition in one day.

15. A method for preparing a solution for oral administration, the method comprising dissolving a powdered composition according to claim 1 and optionally a second powder composition which is a supplement and/or food product in water.

16. A method of use of the kit according to claim 13 as a supplement and/or food product, the method comprising taking from 1 g to 10 g of the first powdered composition in one day.

17. The method of claim 15, wherein the step of dissolving involves dissolving both the powdered composition and a second powder composition which is a supplement and/or food product in water.
